(19) United States
(12) Patent Application Publication
(10) Pub. No.: US 2015/0258336 A1
(43) Pub. Date: Sep. 17, 2015

(54) TREATING AND DETECTING INFECTIOUS DISEASES

(71) Applicant: Dragan Danilo Nebrigic, Austin, TX (US)

(72) Inventor: Dragan Danilo Nebrigic, Austin, TX (US)

(21) Appl. No.: 14/214,266

(22) Filed: Mar. 14, 2014

(51) Int. Cl. A61N 1/32

(52) U.S. Cl. CPC A61N 1/32

(57) ABSTRACT

A treatment device, system and process for treating one or more biological targets within an environment is provided. In one implementation, for example, a treatment device comprises an antenna comprising a pair of electrodes configured for electrical coupling with an environment comprising biological targets for delivering a first electromagnetic signal to the environment, wherein the first electromagnetic signal is configured to generate ions within the environment for interacting with the biological targets.

### CROSS-REFERENCE TO RELATED APPLICATIONS

This application claims the benefit of U.S. provisional application no. 61/786,128, filed Mar. 14, 2013, which is hereby incorporated by reference as though fully set forth herein.

### BACKGROUND

Pharmaceutical drugs and ointments are the primary treatment modality for infectious diseases. Antibiotics, for example, are not immune to complications, side effects and over-use which leads to immune resistance in the population. As a result, a cost effective therapeutic option that does not cause immune resistance could potentially have widespread utilization across many infectious diseases.

In the case of nail fungus (onychomycosis), pharmaceutical oral medication such as Griseofulvin, Fluconazole are the medications of choice for treatment. These medications have a modest clinical efficacy rate of 10% as reported in clinical trials. New technologies are required as alternative treatment options to address this chronic problem. Phototherapy provided by lasers is the only device modality currently available. The phototherapy lasers use “bulk heat” as a mechanism of action. This procedure is painful since lasers are color sensitive, the treatment targets are not clear and focused, and visual detection for the treatment location is inaccurate. As a result, lasers currently have a limited application in the treatment of (onychomycosis) and other topical infections.

### BRIEF DESCRIPTION OF THE DRAWINGS

FIG. 1 shows an example environment in which biological targets are present.

FIG. 2 shows an example implementation in which an environment comprises a plurality of colloidal particles each having a positive charge or a negative charge.

FIG. 3 shows an example implementation in which an electrical double layer is formed within an environment via an application of an electromagnetic signal to a pair of electrodes and coupled to the environment.

FIG. 4 shows an example of a system for targeting nail fungus in a toe.

FIG. 5 shows an example implementation of a contact approach of a particle targeting device in which a pair of electrodes are arranged around a toe.

FIG. 6 shows an example implementation of a non-contact approach of a particle targeting device in which a pair of electrodes are arranged around a toe.

FIG. 7 shows an example schematic circuit diagram of an ultrasound pulser programmable logic device.

### DETAILED DESCRIPTION

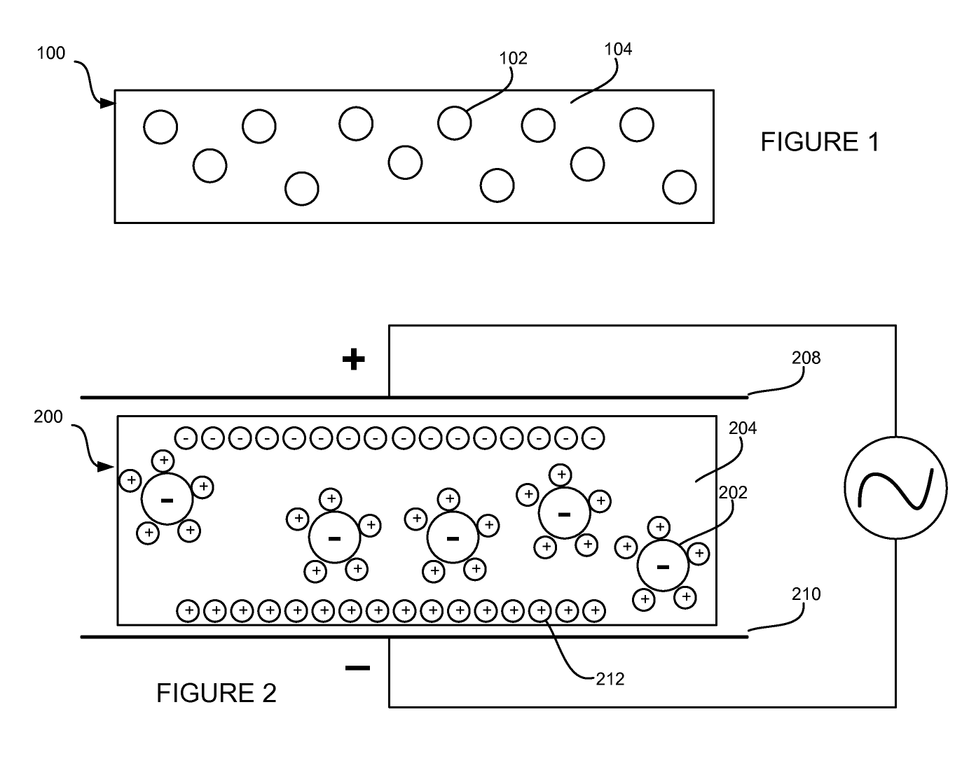

FIG. 1 shows an environment 100 in which biological targets 102 (e.g., fungi, bacteria, or viruses) are present. The biological targets 102, for example, may be present as colloidal particles in a “suspension” of physiological fluids 104 (e.g., a viscoelastic fluidic suspension such as an interstitial fluid that flows between tissue cells 106 of humans and other animals). The biological targets may comprise charged or uncharged colloidal particles. A charged biological target 102, for example, may be positively charged or negatively charged. Bacteria, for example, are typically negatively charged particles. Fungi and viruses, for example, also have various charges.

FIG. 2 shows an example implementation in which an environment 200 comprises a plurality of colloidal particles 202 each having a positive charge or a negative charge. The plurality of colloidal particles 202, for example, may have invaded a tissue viscoelastic fluidic suspension 204. The suspension, for example, may be present in a physiological system, such as in a matrix of an infected nail, an infected wound, acne, or the like.

In the implementation shown in FIG. 2, a pair of electrodes 208 and 210 is provided adjacent to the environment 200 for electrically coupling with the environment 200. In one implementation, for example, a low frequency electromagnetic signal is applied across the pair of electrodes 208 and 210. The electromagnetic signal, for example, may comprise an electromagnetic signal of less than about 500 KHz. In one particular implementation, for example, the electromagnetic signal may be in the range from about 5 KHz to about 200 KHz, although other frequencies are also possible.

The electromagnetic signal generates ions 212 in the environment 200. The ions 212 are free to migrate within the environment 200. In addition to the generated ions 212, intracellular and extracellular liquids contain ions 212 that are also free to migrate within the environment 200 (e.g., within an electric field generated by the electromagnetic signal applied to the electrodes 208 and 210). Where the electrode(s) 208 and 210 are in direct contact with a fluid 204 of the environment 200, ions may be introduced at a transition between the electrode(s) and the fluid 204 adjacent to the electrode(s) 208 and 210. Where the electrode(s) 208 and 210 are not in direct contact with the fluid 204, however, the ions can be introduced by an inductive—capacitive resonant charging process.

The electromagnetic field introduced by the electrodes 208 and 210 creates a “double layer” formed by the ions. Where the electrode(s) 208 and 210 are in direct contact with a fluid 204 of the environment (e.g., through a porous barrier such as a nail), the double layer is created at the transition between the electrode(s) 208 and 210. In the double layer, a surface charge of the electrode(s) 208 and 210 is mirrored by a parallel layer of ions within the fluid 204. The ions in the fluid form a diffuse layer of free ions under the influence of electric attraction and thermal motion.

Where the electrodes are not in contact with a fluid of the environment 200, however, the double layer may be created at a transition in which different layers or objects have different material or electrical properties. A double layer may similarly form at various tissue transitional surfaces (e.g., a nail, a nail matrix or other tissue transition). Thus, a layer of ions within a fluid 204 may minor a parallel surface charge on a tissue within the environment 200.

Ions generated in the environment 200 are also attracted to and surround the charged colloidal particles 202. The ions, for example, may alter a pH of the environment 200 and/or alter a charge of individual colloidal particles 202 within the environment 200. Many biological targets are sensitive to pH and, thus, by creating ions (e.g., hydrogen or hydroxide ions) in the environment the pH within the environment (or within a closely controlled region of the environment) may be controlled to create an environment inhospitable to a particular type of target particle. Depending upon the target, pH can be controlled in situ to provide an inhospitable environment for the target. Thus, a pH of an environment may be controlled to be more acidic or basic depending on a particular target.

In addition, a charge of the colloidal particles 202 (e.g., proteins, bacteria, and fungus) within a suspension provides for reciprocal repulsion of the particles that keeps those particles in suspension. A loss of charge, however, can reduce the repulsive forces of the colloidal particles 202 (e.g., biological targets) which, in turn, can lead to clotting and precipitation of the particles within the physiological fluid.

Charged particles (e.g., charged colloidal or other charged target particles) within the environment can also be detected, controlled (e.g., oriented or displaced), and/or treated through an electrical coupling (e.g., capacitive or inductive coupling) and/or electroacoustics via the electrodes 208 and 210 (or another set of electrodes or antennas). A signal applied to the pair of electrodes 208 and 210, for example, can be used to couple the electrodes 208 and 210 to environment 200 to provide an electrostatic or electromagnetic charge in the environment 200 in which the targets such as the charged colloidal particles 202 reside.

In one particular implementation in which a double layer is formed within the environment 200, for example, the double layer can be used to localize the targets such as charged colloidal particles 202 in a particular region of the environment. The particles 202 localized within region, for example, may be easier to treat by virtue of the region in which they are localized. Nail fungus targets, for example, may be able to be localized within a nail bed under a nail and away from a root of the nail so that they may be more easily treated (e.g., via a laser or microwave bulk heating approach). In addition, the localized particles 202, may also be more effectively treated simply by their proximity to each other (e.g., a given treatment may be more effective since the particles 202 are localized together for treatment and a higher percentage of the targets 202 may be treated with the same treatment technique).

Coagulated target particles, for example, can be targeted for treatment, such as with energy to heat the targeted particles (e.g., bulk heating), a modulated signal (e.g., an amplitude modulated radio frequency signal superimposed on a “charging” direct current signal), electroacoustic energy, or any other targeted treatment methodology. As described above, for example, a pH of the environment 200 can controlled creating ions within the environment (e.g., within a specific region of the environment). In addition, positive ions of a target particle may be “pulled off” the target particle so that the particle will not spread and can be destroyed. Once damaged and/or isolated, a target particle may be destroyed through bulk heating and/or through other methodologies. In one implementation, for example, electrostatic, electroacoustic, and/or electrokinetic forces may be used to compromise a cellular wall of a target particle.

In another implementation, the environment 200 is charged using electrostatic energy and/or the modulated signal described above, a specific ultrasound frequency signal is also applied to the environment 200. The ultrasound frequency signal may be applied to the environment 200 via the pair of electrodes 208 and 210 or via another source. The ultrasound frequency causes motion of a complex “target-external ion” (e.g., a super-ion) and electrical current is generated in the environment 200 due to mechanical motion of the target-external ion. The generated electrical current, in turn, can provide a local voltage breakdown between the target-external ions other close target-external ions to create mechanical destruction of the target particles (e.g., destruction of a target fungus stem at a location where the fungus is tethered, comprising a cellular wall of a target particle, or the like). In this particular implementation, heating caused by the electroacoustic generated current can further affect free particles within the environment 100 (e.g., free fungus spores that have broken off the target fungus stems).

In yet another implementation, target particles 202 may be exposed to broadband light to destroy or otherwise harm the target particles 202. Where the target particles 202 have been localized, for example, broadband light may be directed onto the localized target particles 202. Similarly, where motion of the target particles 202 is controlled, broadband light may be directed into a path through which the target particles 202 will move to destroy or otherwise harm the target particles 202. In addition, where target particles 202 are free floating in the environment 200 (e.g., spores broken off stems of fungus), the free floating particles 202 may be exposed to broadband light within the environment to destroy or otherwise harm the target particles 202. Where a fungus spore has broken off of stems of the fungus, for example, exposure of the spores to broadband light may prevent the spore from starting another stem root at the other location as well where the light could affect any superficial fungus, such as a surface of a nail plate or a mission nail plate location.

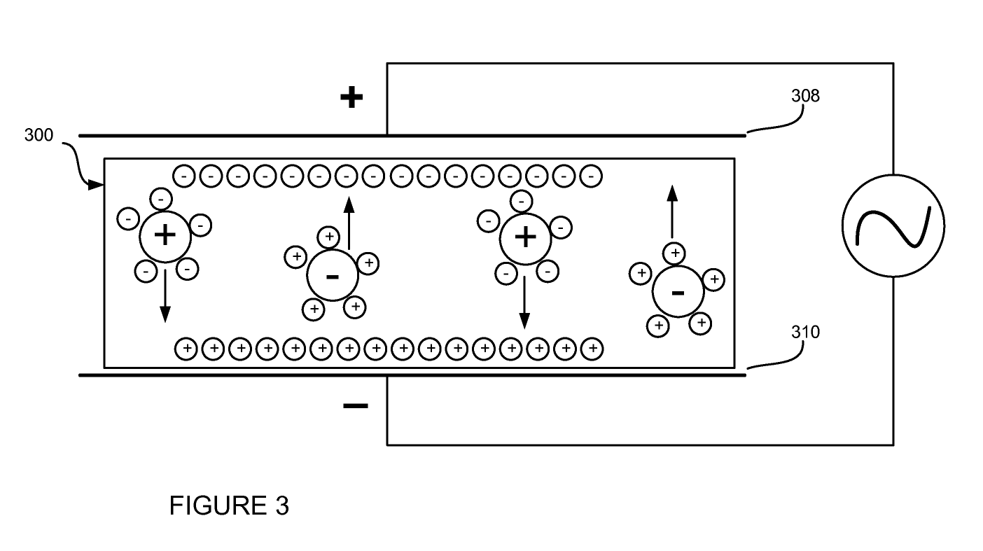

FIG. 3 shows an example implementation in which an electrical double layer is formed within an environment 300 via an application of an electromagnetic signal to a pair of electrodes 308 and 310 coupled to the environment. In this implementation, the electrical double layer is created proximal to a transitional surface in or adjacent to the environment 300. A transitional tissue within the environment 300, for example, is electrically charged and ions within the environment surround one or more target particles within the environment to electrically charge the particles to an isoelectric point. At an isoelectric point, a colloidal system is least stable from a zeta potential standpoint. The isoelectric point is related to a specific pH value for which the zeta potential is equal to 0 mV.

Clustering groups of combined particles (e.g., target particles and surrounding ions) to larger groups of particles creates gradients of materials with a more substantial electrical capacitive difference relative to a surrounding tissue (i.e., colloidal islands). The colloidal islands can then be exposed to higher frequencies (e.g., between 1 MHz to 2.4 GHz or similar frequencies) where Maxwell-Wagner conditions are dominant by selectively heating the colloidal islands alone. In addition, the colloidal islands can have pH values modified locally for a short time period to a pH value(s) that provide unfavorable living conditions for a particular living target particle. At the same time, the colloidal islands can be exposed to electrical force based vibration where the particle walls are compromised to destroy or damage the target particles within the colloidal islands.

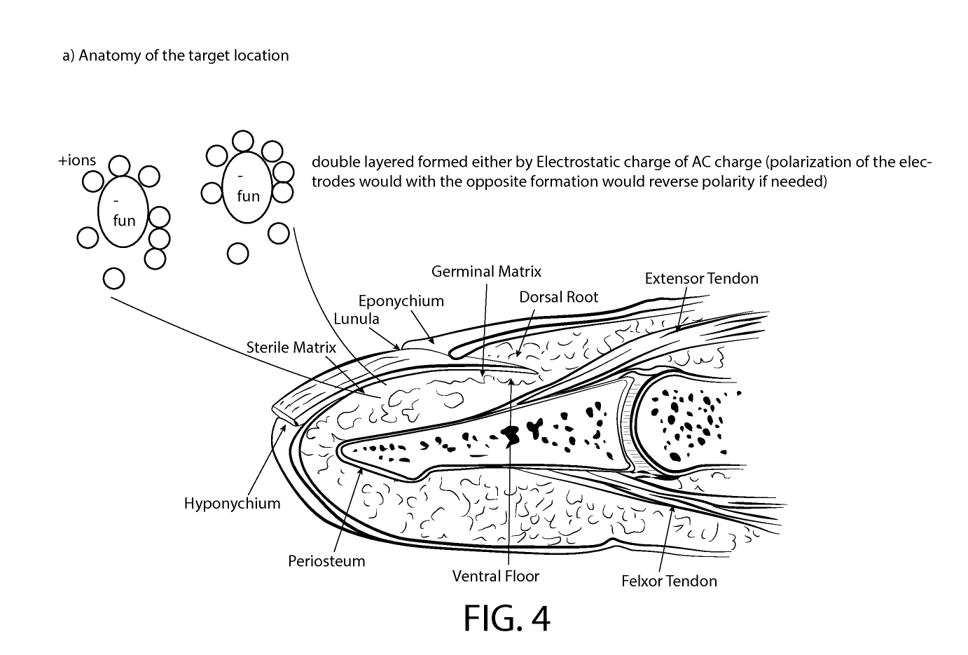

FIG. 4 shows an example of a system for targeting nail fungus in a toe. The system comprises a pair of electrodes that forms a double layer under a toenail. In the implementation shown in FIG. 4, a target particle comprises a negatively charged fungus particle, although any other target particle having a positive or negative charge may be used. Where a positive charged target particle is used, for example, the polarization of the electrodes may be reversed to reverse the charge of the double layer within the toenail environment.

The double layer, for example, may be formed by an electrostatic charge and/or by an alternating current (AC) charge. The alternating current charge, for example, may comprise an amplitude modulated radio frequency superimposed on an intermittent charging direct current signal.

In this particular implementation, the double layer comprises positively charged ions that are attracted to and interact with the negatively charged fungus target particles. As shown in FIG. 4, the fungus target particles are each surrounded by a plurality of positively charged ions generated by the electrostatic charge and/or alternating current charge. As described above with respect to FIG. 1, the double layer field may be used to move, align, or locate the fungus target particles to aid treatment of the target particles. Further, the positive ions increase the ionization of the environment in which the toe is exposed.

The increased ionization may be used to alter a pH of the toenail environment to increase the hostility of the toenail environment to the targeted fungus particles or to otherwise increase an effectiveness of the treatment of the targeted fungus. As described above, the increased ionization of the environment can decrease a charge of target particles within a suspension to reduce repulsion between the particles and encourage clotting and precipitation of the target particles. Once the target particles are coagulated within the sterile or germinal matrix of the toe, the target particles may be treated in any number of ways, such as by breaking down the targeted particles (e.g., with electrostatic or AC energy), exposure to electromagnetic vibration to compromise a cellular structure of the targeted particles, electroacoustic current generation created by motion of ionic targets in suspension (e.g., a physiological fluid such as a interstitial fluid), a current induced cellular wall breakdown, application of broadband light, bulk heating, and/or the like.

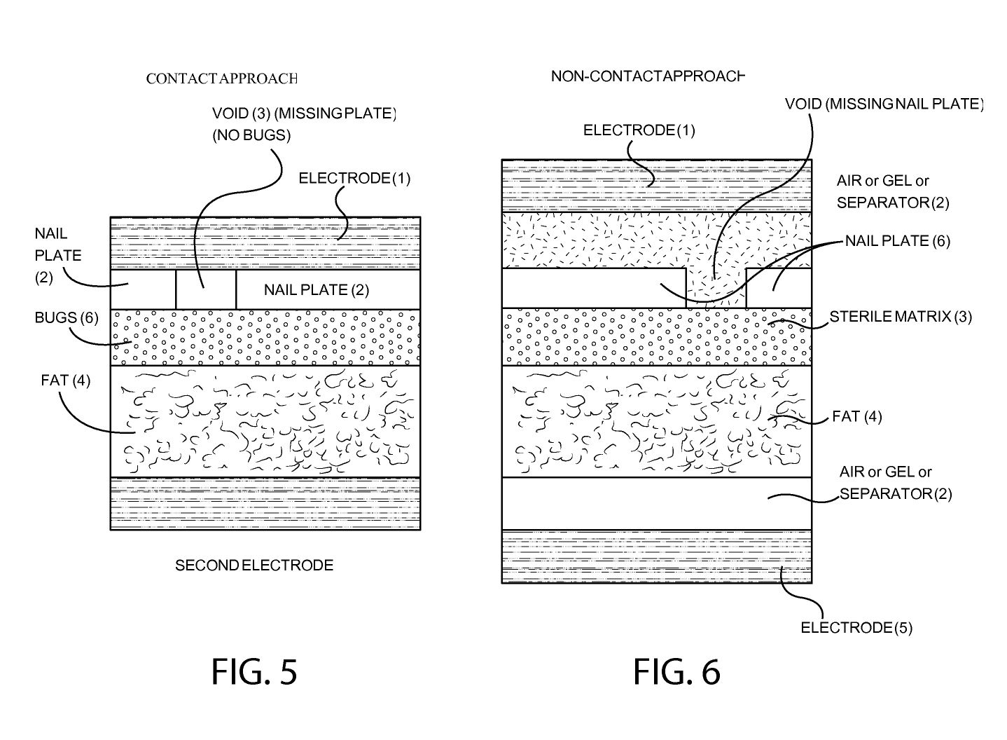

FIG. 5 (labeled “Contact Approach”) shows an example implementation of a contact approach of a particle targeting device in which a pair of electrodes are arranged around a toe. In this particular implementation, a first electrode is positioned directly adjacent to a nail plate of a patients toenail. The second electrode is electronically coupled to the first electrode and is arranged to provide an electrical field within a patitent, such as a sterile matrix and/or a germinal matrix of a nail bed of a toenail. In FIG. 5, the nail plate is shown having a void where the toenail had fallen off due to a fungus infection of the toenail.

FIG. 6 (labeled “Non-Contact Approach”) shows an example implementation of a non-contact approach of a particle targeting device in which a pair of electrodes are arranged around a toe. In this particular implementation, a first electrode is separated from the nail plate of the patient by a layer of air, gel, or other separator. The second electrode is electronically coupled to the first electrode and is arranged to provide an electrical field within a patient, such as a sterile matrix and/or a germinal matrix of a nail bed of a toenail, through the layer of air, gel, or other separator material.

In FIG. 6, the nail plate is shown having a void where the toenail had fallen off due to a fungus infection of the toenail. In this particular implementation, the conductive gel or other separator may extend into the void to make a better connection with the sterile and/or germinal matrix of the toenail.

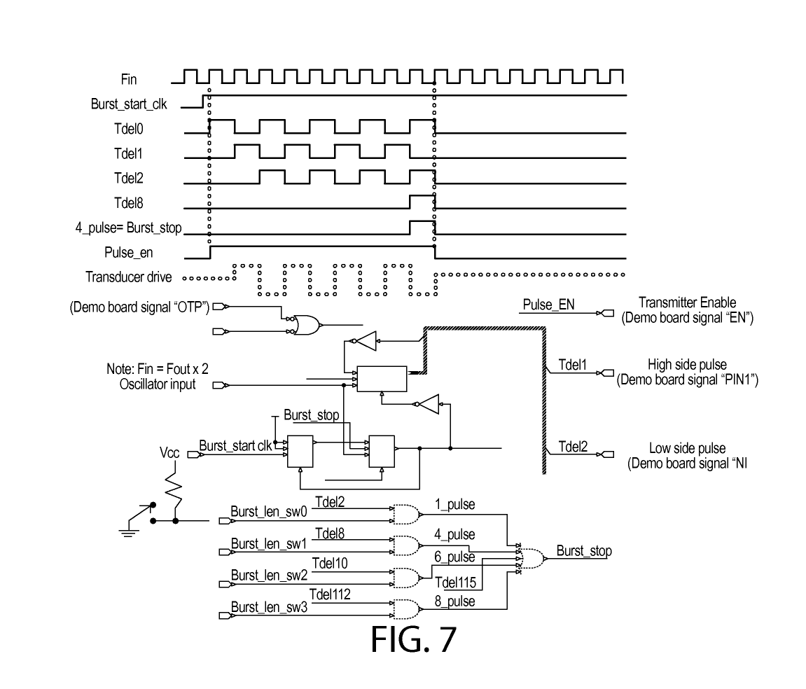

FIG. 7 illustrates an example schematic circuit diagram of an ultrasound pulser programmable logic device.

The embodiments of the invention described herein are implemented as logical steps in one or more computer systems. The logical operations of the present invention are implemented (1) as a sequence of processor-implemented steps executing in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, it should be understood that logical operations may be performed in any order, unless explicitly claimed otherwise or a specific order is inherently necessitated by the claim language.

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

1. A treatment device comprising:
an antenna comprising a pair of electrodes configured for electrical coupling with an environment comprising biological targets for delivering an electromagnetic signal to the environment, wherein the electromagnetic signal is configured to generate ions within the environment for interacting with the biological targets.

2. The treatment device of claim 1 wherein the electromagnetic signal is adapted to generate an electrical double layer of ions within a fluid of the environment.
